(19) United States
(12) Patent Application Publication
(10) Pub. No.: US 2024/0287439 A1
(43) Pub. Date: Aug. 29, 2024

(54) SYSTEMS AND METHODS FOR MANUFACTURING CELLS

(71) Applicant: SOTIO BIOTECH INC., Boston, MA (US)

(72) Inventor: Geoffrey L. Hodge, Sutton, MA (US)

(21) Appl. No.: 18/682,648

(22) PCT Filed: Aug. 11, 2022
(86) PCT No.: PCT/US22/40113

§ 371 Date: Feb. 9, 2024

Related U.S. Application Data
(60) Provisional application No. 63/231,994, filed on Aug. 11, 2021.

(51) Int. Cl. C12M 1/36; C12M 1/00; C12M 1/12

(52) U.S. Cl. CPC C12M 41/48; C12M 29/00; C12M 37/00; C12M 41/14

(57) ABSTRACT

Systems for semi or fully automated non-parallel random access manufacturing of cells comprising (a) an incubator arranged to house multiple cell culture vessels, and (b) one or more connection interfaces, each of which comprises (i) a first connector, and (ii) a first sterilizable space, wherein the first connector is operable to connect a first container and a cell culture vessel in the first sterilizable space. Also provided herein are methods for manufacturing cells using the systems provided herein.

### CROSS REFERENCE TO RELATED APPLICATIONS

This application claims the benefit of the filing dates of U.S. Provisional Application No. 63/231,994, filed Aug. 11, 2021, the entire contents of which is incorporated by reference herein.

### FIELD

This application relates to systems and methods for manufacturing cells, e.g., manufacturing a cell therapy such as immune cells expressing a chimeric antigen.

### BACKGROUND

Adoptive cell therapy, e.g., chimeric antigen receptor (CAR)-T cell-based therapy, is becoming a promising option for treating various types of cancer because of its potential to evade genetic and cellular mechanisms of drug resistance, and to target tumor cells while sparing normal tissues. Clinical manufacturing of high-quality therapeutic cells is a prerequisite for the wide application of this technology.

Current approaches for producing therapeutic cells for use in adoptive cell therapy typically involve ex vivo enrichment, activation, and expansion of T cells and genetic modification of the T cells using retroviral or lentiviral vectors to introduce an exogenous nucleic acid coding for a chimeric receptor. This whole process is time consuming and expensive. It is of great interest to develop systems and methods that enable efficient, high-volume, and cost-effective production of therapeutic cells.

### SUMMARY OF THE INVENTION

Provided herein are systems and methods for non-parallel random access manufacturing of cells in a manner that allows multiple cell culture vessels to undergo different steps of the manufacturing process at the same time. The system for non-parallel manufacturing of cells as disclosed herein enables the automated manufacture with multiple cell culture vessels handled independently. Adoptive cell therapy is typically a personalized therapy based on immune cells isolated, e.g., by leukapheresis, from patients and individually processed. Accordingly, starting materials for the manufacture of the cell culture inherently vary, and manufacturing processes are difficult to automate and are done manually, thereby increasing costs for such therapies and the risk for failure to successfully produce such therapy due to human error. The present disclosure provides systems and methods for independent/non-parallel handling of cell culture vessels in various manufacturing operations, whereas such manufacturing operations can be performed under sterile conditions and/or can be partially or fully automated.

In a first embodiment, the invention relates to a system for non-parallel random access manufacturing of cells, wherein the system comprises: one or more incubator arranged to house a plurality of cell culture vessels, wherein each cell culture vessel is configured for moving in and out of the incubator(s) independently; (b) one or more workstations configured to host each of the cell culture vessels to perform one or more manufacturing operations; and (c) a transfer device for moving the cell culture vessels between two incubators, between the incubator and the workstation, or between two workstations, wherein the transfer device of (c) operates automated, manually, or a combination thereof.

In a second embodiment, the invention relates to the system further comprising a controller, the controller includes (i) a processor, (ii) a memory storing manufacturing operations, sampling and instructions, when executed by the processor, cause the processor to schedule movements of the cell culture vessels between the incubator(s) and the workstations, wherein the movements are configured to execute automatically.

In a third embodiment, the invention relates to method for non-parallel processing of multiple cell cultures, the method comprising (i) providing the system for non-parallel random access manufacturing of cells, wherein the system comprises multiple cell culture vessels, each of which comprises a cell culture, and (ii) performing manufacturing operations on one or more of the cell cultures in the multiple cell culture vessels, wherein operates automated, manually, or a combination thereof as disclosed herein. In some embodiments, a connection interface for sterile connection and liquid transfer between one or more of the cell culture vessels and a bioprocess container.

The details of several embodiments of the invention are set forth in the accompanying Figures and the Detailed Description. Other features, objects, and advantages of the invention will be apparent from the description and from the claims.

### BRIEF DESCRIPTION OF THE DRAWINGS

Various aspects and embodiments will be described with reference to the following figures. The figures are not necessarily drawn to scale.

FIG. 1 is a schematic depiction of an exemplary system for non-parallel manufacturing of cells comprising an incubator, a connection interface and workstations. Any of the cell culture vessels may be moved to a workstation (e.g., a centrifuge for separation) and/or to a connection interface. A solution (e.g., media, buffers, and vectors) may be transferred from a container to the cell culture vessel via the connection interface. Alternatively, or in addition to, samples may be taken from the cell culture vessel via the connection interface. The samples may be analyzed at workstations (e.g., a flow cytometer and a cell counter for analytics).

FIGS. 2A-2B are schematic depictions of an exemplary connection interface 100 comprising a first container 110, a connector 120, and a housing 130 forming a sterilizable space 140 for sterile connection and liquid transfer between a second container 150, e.g., a culture vessel, and the first container 110 via the connector 120, in accordance with some embodiments of the technology described herein. FIG. 2A shows the connector 120 attached to a connector (e.g., a septum) 160 of the second container 150 (e.g., a culture vessel) and a fluid conduit 170 of the first container 110. FIG. 2B shows the connection interface of FIG. 2A further comprising a sterilizer 180, and a first container 110 that further comprises a pinch clamp or valve 190.

FIGS. 3A-3C are schematic depictions of an exemplary connection interface for sterile connection and liquid transfer via tube welding, in accordance with some embodiments of the technology described herein. FIG. 3A shows a connection interface 100 within a sterilizable space 140 comprising weld heads 200a, 200b, 200c, 200d for welding the fluid conduit 170a of the second container 150 such as a culture vessel and the fluid conduit 170b of the first container 110 via a connector such as tubing 210 having a removable tube portion 500 (e.g., an intermediate spool). FIG. 3B shows the connection established between the second container 150 (e.g., a culture vessel) and the first container 110. FIG. 3C shows the initial positioning of tube weld with auto-loading weld mounts configured for an intermediate/spool piece (500). Weld mounts (310) hold source container tubing (115) with destination container tubing (215), e.g., culture vessel tubing, via spool piece/intermediate portion of tubing (500). 110—first container such as a source container, 150—second container such as a destination container, for example, a culture vessel.

FIGS. 4A-4F are schematic depictions of an exemplary connection interface, in accordance with some embodiments of the technology described herein. 105—connection assembly, 170a, 170b—fluid conduit, 190, 190a, 190b—valves, 220a, 220b—connectors, 260—intermediate piece of tubing, 230, 230a, 230b—seals, 240a, 240b—spaces, 250a, 250b—ports. FIG. 4A and FIG. 4B: schematic depictions of two connectors before (FIG. 4A) and after partial connection to create a sterilizable space across the connection interface (FIG. 4B). FIGS. 4C-4E are schematic depictions of an exemplary connection interface before connection (FIG. 4C), after partial connection to create a sterilizable space across the connection interface(s), (FIG. 4D) and, fluid flow through after full connection (4E). FIG. 4F is a schematic depiction of an exemplary connection interface in a sterilizing chamber.

FIGS. 5A-5F are schematic depictions of an exemplary process for sterile connection and liquid transfer, in accordance with some embodiments of the technology described herein, including load component (FIG. 5A), sterilize components (FIG. 5B), make connection (FIG. 5C), transfer liquids (FIG. 5D), break connection (FIG. 5E), and eject components (FIG. 5F).

FIGS. 6A-6B are schematic depictions of an exemplary process for manufacturing cells, in accordance with some embodiments of the technology described herein. FIG. 6A is an exemplary process for cell culture, passaging, and expansion. FIG. 6B is an exemplary process for manufacturing cells transduced with a viral vector.

FIG. 7 is a schematic depiction of a cell manufacturing system controlled by a computer system.

FIG. 8 is a schematic depiction of the positioning of a pump (600) and pinch valves for tube coupling between a first container 110 such as a source container and a second container 150 such as a destination container, which may be a cell culture vessel via an intermediate/spool piece (500).

### DETAILED DESCRIPTION OF THE INVENTION

The present disclosure is based, at least in part, on the development of systems and methods for non-parallel manufacturing of cells, for example, for manufacturing cell therapeutics. The systems and methods disclosed herein led to at least the following advantageous outcomes:
(a) High-volume production of manufactured cells resulting from systems and methods that enable manufacturing cells in a non-parallel manner in which multiple cell culture vessels undergo different steps of the manufacturing process at the same time. Such non-parallel processing allows efficient use of cell manufacturing equipment, which otherwise may be idle when manufacturing cells in a parallel manner. The systems and methods disclosed herein also are easy to automate, which increases production rates and productivity.
(b) Decreased lapse in production of manufactured cells resulting from systems comprising components that are easily accessible for repair and/or replaceable if improved technology becomes available.
(c) Efficient and variable scale production of multiple cell therapies resulting from systems and methods that are flexible, adaptable, and/or scalable to meet a wide range of production demands.
(d) Reduced risk of cross contamination between samples resulting from systems and methods that utilize components that may be decontaminated between uses or disposed of after a single use.

Accordingly, provided herein are systems and methods for non-parallel manufacturing of cells, e.g., manufacturing cell therapies such as T cells expressing a chimeric antigen receptor (CAR).

### I. Systems for Non-Parallel Random-Access Manufacturing of Cells

Conventional system for manufacturing of cells involves a sequential set of manufacturing process steps in a clean room or a laboratory space that houses machinery, plastic ware, cell culture, glassware etc. that may be operated manually, automatically or in combination. The manufacturing process steps are performed by skilled research workers and/or technicians and is labor-intensive. Further, manual operation significantly increases the risk of contamination. Manual manufacturing process steps are robust that includes initiation, maintenance, analysis and/or trouble-shooting but is prone to variability and is difficult during implementation in the scale-up phases. Eventually, this led to the establishment of an automated system for manufacturing of cells especially for large-scale productions. Individual closed systems or a machine for cell therapy may be used in series (i.e., end-to-end running of an entire manufacturing process as a single batch) or in parallel where modules of similar manufacturing process steps run simultaneously. Both these closed system configurations led to reduced risk of contamination.

End-to-end closed systems are efficient and reliable for a small batch or during early discovery phase of a cellular product. Nevertheless, they have some disadvantages such as long set-up time, inefficient usage of sub-system equipment (i.e., at a given time point only some parts are being utilized), batch-to-batch variation, lacks design flexibility and would manufacture only one product at a time. These disadvantages led to the ushering of a more tenable system conducting similar or identical manufacturing process steps in a parallel way. Such parallel systems may be fully automated that includes a controller, communication interface (for e.g., scheduling software, pre-stored programs) and multiple transfer devices (for e.g., robotic arm). Parallel systems for manufacturing of cells may have higher utilization rates, shorter processing output (i.e., elimination of production bottlenecks), flexibility and may ensure repeatability & traceability.

To ensure reliable, sterile and potent cellular products, all manufacturing process steps undergo several sampling interventions. Continuous sampling interventions optionally comprises cell count, cell viability, level of transduction (e.g., via flow cytometry and/or PCR), growth medium properties (e.g., pH, osmolality, and/or metabolites), contaminants (e.g., BSA, DNA, etc. which may be determined during wash steps), or a combination thereof. Therefore, a more flexible system which can allow for process adjustments based on process data may be more advantageous.

The present disclosure provides a system for manufacturing cells (e.g., therapeutic cells) comprising: (a) one or more incubators arranged to house multiple cell culture vessels, each being configured for moving culture vessels in and out of the incubator(s) independently, (b) one or more workstations for hosting each of the cell culture vessels to perform one or more manufacturing operations; and (c) a means of moving the cell cultures between one of the incubator(s) to one of the workstations or between the workstations. In some instances, wherein the means of (c) can be a device, fixture and/or structure that operates automated, manually, or a combination thereof. For example, the means can be a transfer device (e.g., a robotic arm) moves the cell culture vessels. In some embodiments, the transfer device moves the cell culture vessels between two incubators. In some embodiments, the transfer device moves the cell culture vessels between two workstations. In some embodiments, the transfer device moves the cell culture vessels between an incubator and a workstation.

Non-parallel in the context of this invention shall mean that during the manufacturing of cells, multiple cell culture vessels containing the cells may be processed individually at different times, using different process steps or sequences, depending on certain parameters with the same system. In some embodiments, the multiple culture vessels each host a cell culture, e.g., from one patient, and wherein one or more, but not all, manufacturing operations on the cell cultures in the multiple cell culture vessels are performed simultaneously.

Random access in the context of this invention shall mean the ability to pair any individual cell culture with any unit operation (e.g. sampling, fluid transfer, etc.) at any time required by its individual process program and parameters (e.g., cell count, cell viability, level of transduction, growth medium properties such as pH, oxygen, temperature).

The invention further provides for solutions for sterile connection and liquid transfer using a connector system with a first and second connector defining a sterilization chamber as further described herein, and a tube welding method as further described herein that allows for multiple sterile connection and detachment in an automated fashion and that are preferably integrated into the system for non-parallel random-access manufacturing of cells. See, e.g., International Application No. PCT/US22/31764, the relevant disclosures of which are incorporated by reference for the subject matter and purpose referenced herein.

Accordingly, in some embodiments, the system is configured for sterile connection and liquid transfer between the cell culture vessels and one or more bioprocess containers. In addition, the invention provides for cell culture vessels that are especially suitable for the non-parallel random-access manufacturing of cells due to customizable design for measuring and cell manipulation steps as further described herein. In some embodiments, the bioprocess container comprises media bags, buffer bags, sample containers, waste containers or a combination thereof. See, e.g., International Patent Application No. PCT/US2022/032426, the relevant disclosures of which are incorporated by reference for the subject matter and purposes referenced herein.

Any incubator suitable for housing multiple cell culture vessels may be used in systems disclosed herein. The incubator may be any suitable shape or size, and set to any suitable temperature.

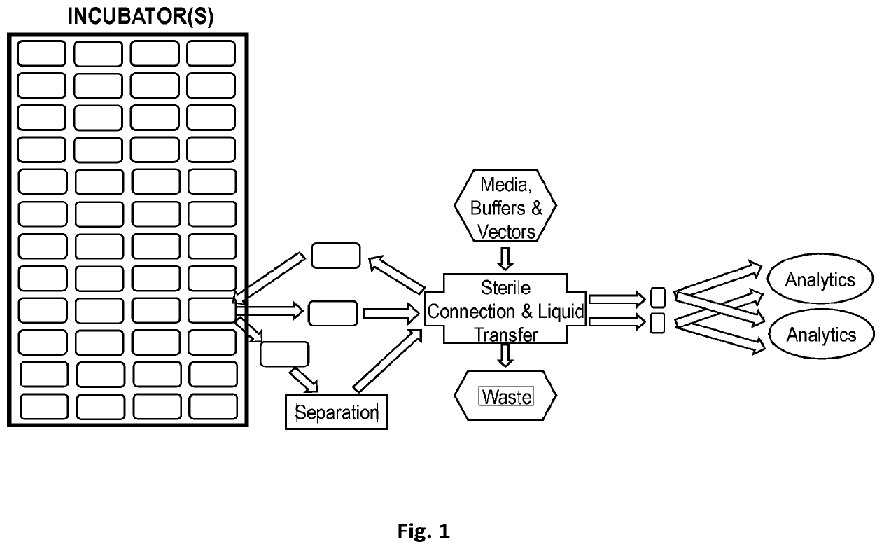

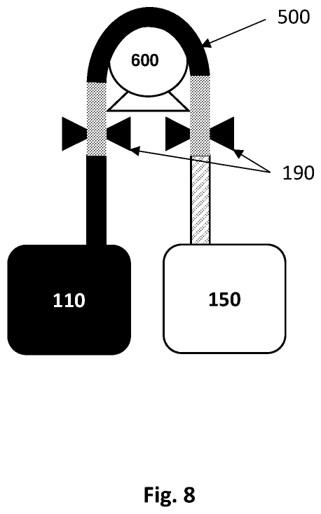

As shown in FIG. 1, an exemplary system for non-parallel manufacturing of cells may comprise one or more incubators and one or more workstations. In some embodiments, one or more incubators or workstations comprise a connection interface for sterile connection and liquid transfer. In exemplary embodiments, the connection interface comprises (i) a first connector; (ii) a second connector, wherein the first connector and the second connector define a sterilization chamber comprising a gap between the first connection surface and the second connection surface; preferably, wherein the gap is an enclosed space accessible through at least one opening, preferably optionally a port; wherein the gap optionally comprises a sterilization agent; wherein the first connector is fluidically coupled with a first container and the second connector is fluidically coupled with a second container; (iii) a first sterilizable space, which optionally comprises a sterilization agent, wherein the first connector is fluidically coupled with a first container and the second connector is fluidically coupled with a second container in the first sterilizable space; and (iv) a liquid transfer device including one or more pump (for e.g., peristaltic) and one or more valves (for e.g., pinch valve) configured to facilitate liquid transfer between the first container and the second container to avoid back contamination (e.g., as depicted in FIG. 8).

In other embodiments, transferring a liquid between the first container and the second container comprises: (i) interlocking one or more (pinch) valves and/or a (peristaltic) pumps; (ii) slightly rotating the peristaltic pump to create a positive (i.e. injection on one side of the peristaltic pump) or a negative pressure (i.e. suction on the other side of the peristaltic pump) in one of the first tube or the second tube prior to releasing the one or more pinch valves to cause a positive or a negative pressure in one of the first tube or the second tube; (iii) rotating a peristaltic pump between two interlocking valves prior to activating the interlocking valves; and/or (iv) pumping the liquid from the first container to the second container or pumping the liquid from the first or second container to the third container.

A workstation, as used herein, refers to a device for performing one or more processes involved in manufacturing cells, e.g., culturing, processing, analyzing, and/or handling cells and/or reagents involved in manufacturing cells. Examples of workstations include a workstation for cell expansion (a cell expansion workstation), a workstation for cell separation (a cell separation workstation, e.g., a centrifuge), a workstation for cell analysis (a cell analysis workstation, e.g., a flow cytometer), and a workstation for cell imaging (a cell imaging workstation, e.g., a cell counter). In some embodiments, the one or more manufacturing operations comprise centrifugation, mixing, media removal, media addition, feed addition, vector addition, sampling, buffer addition, buffer removal, or a combination thereof. In some embodiments, the one or more workstations are configured for sterile connection and liquid transfer between the cell culture vessels and one or more bioprocess containers. In some instances, the bioprocess containers comprise media bags, buffer bags, sample containers, waste containers, or a combination thereof.

A workstation may perform one or more processes involved in manufacturing cells on the cell culture vessel or a sample therefrom. For example, in FIG. 1, a workstation may be a cell separation workstation such as a centrifuge in which the cell culture vessel is placed, and cells are separated. In another example, in FIG. 1, workstations may be cell analysis workstations such as flow cytometers and/or cell counters in which samples from the cell culture vessel are analyzed.

Although the system in FIG. 1 is shown as comprising three workstations, systems disclosed herein may comprise any number of workstations, which perform any of the one or more processes involved in manufacturing cells.

In some embodiments, at least one of the workstations comprise a connection interface for sterile connection and liquid transfer. In some instances, the connection interface comprises: (i) a first connector; (ii) a second connector; (iii) a first sterilizable space, which optionally comprises a sterilization agent, wherein the first connector is fluidically coupled with a first container and the second connector is fluidically coupled with a second container in the first sterilizable space; and (iv) a liquid transfer device including pumps and valves to allow for liquid transfer between the first container and the second container to avoid back contamination.

For example, the connection interface may include a device for sterile connection such as a sterilizer. The sterilizer may include an energy source that directs energy towards the sterilizable space and components placed within that space. The energy source can be heat and/or steam. Alternatively or additionally, the sterilizer may be a sterilizer agent, such as a fluid selected from a gas (e.g., ozone), a sterilizing chemical (e.g., ethanol) or a vapor.

A container for use in a system and/or a connection interface disclosed herein may be any suitable shape or size, and any suitable material. For example, when receiving cells in the cell culture, the container may be a gas permeable material that permits diffusion of gases sufficient for cell viability. Such containers may be suitable for processing the cells, e.g., culturing and/or centrifuging the cells in the container. Alternatively, or in addition to, the container may be disposable to eliminate risks of contamination. Non-limiting examples of a container include a cell culture container (e.g., a cell culture bag or a cell culture flask or a rigid bioreactor), a destination bag, a source bag, vial or syringe (e.g., for adding liquids like media, viral vector suspension or solutions of growth hormones, cytokines, drugs or other compounds to manipulate cells) or a waste container. A destination bag may be used for either receiving the cell culture medium or the cells of the cell culture.

A container for use in a system and/or a connection interface disclosed herein may include a fluid conduit and/or opening for transferring liquid between the first container and the second container. For example, a container may include a fluid conduit, which may be attached to a connector when performing a sterile liquid transfer. Alternatively, or in addition to, a container may include a cannula, a fitting for receiving a cannula, or a septum for transferring liquid under sterile conditions.

A container for use in a connection interface disclosed herein may be any suitable shape or size, and any suitable material. For example, a container may be a cell culture vessel that is disposable to eliminate risks of contamination. A non-limiting example of a cell culture vessel includes a cell culture bag and/or a rigid cell culture vessel with a vent and/or gas permeable membrane.

A cell culture vessel for use in a system and/or a connection interface disclosed herein may be any suitable shape or size, and any suitable material. For example, the cell culture vessel may be disposable to eliminate risks of contamination. A non-limiting example of a cell culture vessel includes a cell culture bag.

A cell culture vessel for use in a system and/or a connection interface disclosed herein may comprise a fluid conduit and/or opening for transferring liquid between the container and the cell culture vessel. For example, the cell culture vessel may comprise a fluid conduit, which may be attached to a connector when performing a sterile liquid transfer. Alternatively, or in addition to, the cell culture vessel may comprise an opening comprising a septum for transferring liquid under sterile conditions.

In addition, the system of the present invention disclosed herein further comprise a culture vessel suitable for use in automated non-parallel manufacturing of cells. In an exemplary embodiment, the cell culture vessel is disclosed, comprises (a) an inner container comprising wherein the pocket defines a volume within which a cell culture is maintained during manufacture of a cell therapy, and (b) an outer shell configured to receive and support the container, wherein the outer shell includes a shell top and a shell bottom that cooperate with one another to form a chamber within which the inner container is disposed, optionally, encapsulated.

In some embodiments, the inventors have recognized that such an inner container may be easily fabricated, e.g., a flexible cell culture bag from thin plastic materials, which may keep costs down for consumers, may increase the ease of getting and using the cell culture vessel, and may simplify the manufacturing process as well as reduce plastic waste. The inner cell culture container may include any container suitable for containing the cell culture (e.g., flexible bags). For example, the inner container may be formed of rigid materials, flexible materials, deformable materials, stretchable materials, or combinations thereof. In some embodiments, the inner container may include an inner bag arranged to contain the fluid. In other embodiments, the inner container may include a rigid frame and one or more film components attached to the frame.

In some embodiments, the inner container may be formed, at least in part, of a gas permeable film to allow oxygen diffusion for cell growth and have one or more conduits for fluid transfer. For example, the inner containers may be disposable, and may be easily loaded into the outer shell during preparation of a first cell therapy, and switched out when a second cell therapy is to be prepared. In turn, such disposable inner container would fit into a reusable outer shell, which provides the support for the bag to transfer the cell culture bag between incubator and work stations.

In other embodiments, the volume of the pocket of the inner container is arranged to maintain the cell culture during non-parallel manufacturing of cells is adjustable, optionally, wherein the outer shell comprises the at least one clamp and the volume of the pocket is adjustable via the clamp, which optionally is a sliding clamp. For example, in some embodiments, the pocket may be arranged to have a smaller volume during the start of manufacturing. In such an example, the volume of the pocket may be increased as manufacturing progresses and the volume of the cell culture increases with cell growth.

In one embodiment, the outer shell fits into the rotor of a centrifuge and thereby can the cell suspensions be directly centrifuged allowing for automation, as the outer shell can be easily grabbed by for e.g., a robot to be transferred into and from the centrifuge. The inventors have further recognized that advantages may be realized, if the cell culture vessel (e.g., culture bag) is well positioned and protected while maintaining sterility of its contents (e.g., cell culture, vector, media etc.). In some embodiments, the outer shell may be arranged to support and/or protect the inner container.

In some embodiments, the outer shell may protect the inner container from puncturing and/or tearing during transport and/or connection of the cell culture vessel (e.g., the assembly) to a workstation. In some embodiments, the outer shell also may provide support for the inner container during processing. For example, the outer shell may provide support when stress is exerted on the container, such as during centrifugation, which may prevent rupturing of the container, or at least a portion of the container. In some embodiments, the cell culture container includes a pocket within which the cell culture is contained during manufacture.

In yet another example, systems disclosed herein may comprise a controller arranged to control each of the components of the system. Any of the systems disclosed herein may further comprise a controller, which schedules movements of the cell culture vessels between the incubator(s) and the workstations. In some examples, the controller may be arranged to control moving the cell culture vessel and/or the connection interface, and/or connecting the connector to the cell culture vessel and the container, and/or transferring liquid between the cell culture vessel and the container. In some examples, the controller may control the temperature and carbon dioxide level of the incubator. In some embodiments, the controller schedules the movements of the cell culture vessels between the incubator(s) and the workstations. In some embodiments, the controller schedules the movements based on calculation of analytical in-process data, which optionally comprising cell count, cell viability, level of transduction (e.g., via flow cytometry and/or PCR), growth medium properties (e.g., pH, osmolality, and/or metabolites), contaminants (e.g. BSA, DNA, etc. which may be determined during wash steps), or a combination thereof.

The controller also may control one or more workstations to process the cell culture. In some embodiments, at least one of the workstations comprise a connection interface for sterile connection and liquid transfer. In such instances, the controller may control workstations based on one or more desired operating parameters. For example, when the workstation is a centrifuge, the controller may direct the centrifuge to run for a desired period of time and speed. In some embodiments, the operating parameters are determined based upon the cell therapy being prepared. As will be appreciated, the operating parameters may vary from cell therapy to cell therapy.

In some embodiments, the controller may be arranged to collect and store data from one or more of the workstations during the manufacturing process. In such instances, the controller may be arranged to process the collected data. The controller also may be arranged to adjust the operating schedule and/or operating parameters of at least one of the workstations based on the feedback from another workstation or analytical in-process data. For example, in some embodiments, the amount of medium or vector added to the cell culture vessel may be based on the cell count. In such embodiments, based on the measured cell count, the volume of medium to be added to the cell culture vessel may be adjusted.

Generally, the controller comprises a processor and memory circuit storing instructions and a processor circuit configured to execute the instructions and/or a memory circuit storing data of the manufacturing operations and sampling. In some instances, the controller schedules the movements of the cell culture vessels between the incubator(s) and the workstations. In some instances, a controller may be programmed to perform the steps automatically. In a preferred embodiment, the controller includes: (I) a processor; (II) a memory storing manufacturing operations, sampling and instructions that, when executed by the processor, cause the processor to schedule movements of the cell culture vessels between the incubator(s) and the workstations, wherein the movements are configured to execute automatically. In some embodiments, the processor is further configured to execute one or more of the following: (i)manage a plurality of cell cultures simultaneously; and (ii) create a custom schedule for the cell culture in each of the cell culture vessels to manage process performance.

In some embodiments, creating the custom schedule for the cell culture is based on pre-programmed instructions, in-process data, scheduling of sequential use of the workstations, or a combination thereof.

In some embodiments, the controller comprises a memory circuit and a processor circuit, the memory circuit storing instructions which, when executed by the processor circuit, cause preceding embodiments to be performed automatically. The system can comprise a computing device (CPU) which can be in communication with a data storage device. In an embodiment, the data storage device can store system data and at least one operating parameter. The data storage can be in the same location as the CPU or at an offsite location wherein the CPU is in telecommunication with the data storage system.

The system can further comprise a plurality of sensors, the sensors can comprise measuring devices that are configured to provide data to the CPU regarding the operation of each component within the system. The sensors displayed in the system may include, without limitation, position sensors, pressure sensors, optical sensors, temperature sensors, force sensors, vibration sensors, piezo sensors, fluid property sensors, time sensors and/or humidity sensors. The system can comprise these sensors to provide data to the CPU to initiate and maintain operation of the system. The data received from the sensors located at the various components of the systems provided data to automate a continuous feedback loop that permits the CPU to maintain and adjust the operation of all components of the system. In a workstation, for example, at a defined timepoint, a controller directs a robot to move a specific container (for e.g., culture bag) comprising a tissue culture (cell container). The robot further puts the inner container within the outer shell. The robot aligns the inner container within interior surface of the rigid cavity of the outer shell. The alignment is carried with respect to openings comprised within the outer shell.

In another embodiment, the timing of media removal and/or media addition may be adjusted to measured metabolites in the medium of the cell culture vessel. In other embodiments, the volume and/or number of buffer washes may be based on analytical data of contaminant removal, pH, or other measured in-process data. In some embodiments, the controller may include a computer or computer system. In some embodiments, the controller may include a tablet or other mobile electronic device (e.g., a mobile telephone). In some embodiments, the controller is connected to one or more workstations and to the incubator. As will be appreciated, the controller may be connected to these devices via any suitable connection, such as via the internet, Ethernet, wireless, Bluetooth, or other suitable connection.

In some embodiments, the controller is operated under a scheduling software. In some instances, the scheduling software may function to manage processing of multiple cell cultures in the cell culture vessels. In some embodiments, the scheduling software is designed to manage dozens to hundreds of cell cultures at the same time. In other embodiments, the scheduling software is designed to create a custom schedule for the cell culture in each of the cell culture vessels to optimize process performance. In a preferred embodiment, the optimization of process performance is based on pre-programmed instructions, in-process data, scheduling of sequential use of the workstations, or a combination thereof. In some examples, the controller may comprise a memory circuit storing data from the manufacturing operations and sampling. In some instances, the processing of the multiple cell cultures may be performed at a pre-determined manner. In other instances, the software may adjust such processing based on in-process data.

In some instances, the system allows for processing of the multiple cell cultures simultaneously. In other instances, the system allows for processing of the multiple cell cultures sequentially. In some examples, the system allows for processing of the multiple cell cultures in an independent manner depending upon factors such as in-process data. In some embodiments, the multiple cell culture vessels each host a cell culture, and the one or more manufacturing operations on the cell cultures in the multiple cell culture vessels are performed simultaneously. In a preferred embodiment, one or more of the cell culture vessels of the plurality of cell culture vessels host a cell culture, and wherein the one or more manufacturing operations on the cell cultures in the plurality of cell culture vessels are performed simultaneously.

Aspects of the present disclosure may involve cell culture vessels that are indexed for tracking in a system for manufacturing cells disclosed herein. For example, the culture vessel may include a tag, chip, label, or other identifier arranged to track the location and progress of the culture vessel in the system. In some embodiments, the identifier may be a visual identifier, such as number or barcode printed on an outside of the culture vessel. In other embodiments, the identifier may include an RFID tag with electronically-stored information. As will be appreciated, any suitable identifier may be used to track the culture vessel in a system disclosed herein. In some embodiments, the system is arranged to read and decode the tag (e.g., scan the barcode and/or read the RFID tag) when the cell culture vessel reaches a workstation and/or a connection interface.

The system also may be arranged to read the tag when the cell culture vessel is leaving the workstation and/or the connection interface (e.g., at exit). In this regard, the workstation and/or the connection interface may include a reader for reading the identifier (e.g., tag) on the cell culture vessel. When the system comprises a robotic device, the robotic device may include a reader for reading the identifier.

In some embodiments, the identifier is printed on, embedded in, or otherwise integrally formed with the culture vessel. In other embodiments, the identifier may be attached to the culture vessel before placement in the incubator. For example, the tag may be attached to a protective cage within which the culture vessel is placed before the culture vessel is inserted into the incubator. In other embodiments, the tag may be placed on a coupler (e.g., a band or clip) that may be attached to the vessel. As will be appreciated, in embodiments in which the system is automated, the controller may direct one or more robotic devices to perform steps such as moving the cell culture vessel to the different workstations. The controller also may collect and evaluate data during processing and store the generated data linked to the identifier. In some embodiments, one or more of the process steps may be skipped or altered depending upon dynamic feedback.

In some embodiments, any of the systems disclosed herein can be in an automated setting. In some embodiments, the system may be operated manually. In other embodiments, the system may comprise both automated features and manual features.

Alternatively or in addition, the systems disclosed herein may comprise any number of connection interfaces for sterile connection and liquid transfer. In such instances, the connection interfaces may comprise any suitable number of containers and/or connectors. In some embodiments, the system may comprise a connection interface comprising one or more containers and one or more connectors for sterile connection and liquid transfer between a cell culture vessel and the one or more containers via the one or more connectors. In such instances, the one or more containers and the cell culture vessel may be connected via the one or more connectors in the same sterilizable space or in different sterilizable spaces.

In some embodiments, the system may comprise a connection interface comprising a single container and a single connector for sterile connection and liquid transfer between a cell culture vessel and the container via the connector. In such instances, the container may comprise a cell culture medium or a solution for transferring into the cell culture vessel, which comprises a cell culture, or the container may be a destination bag for receiving either the culture medium or the cells of the cell culture.

In some embodiments, the system may comprise a connection interface comprising a first and a second container and a first and a second connector for sterile connection and liquid transfer between the cell culture vessel and the first container via the first connector, and between the cell culture vessel and the second container via the second connector. In such instances, the first container may comprise a cell culture medium for transferring into the cell culture vessel, which comprises the cell culture, and the second container may be a destination bag for receiving either the culture medium or the cells of the cell culture.

In some embodiments, the system may comprise a connection interface comprising a first, a second, and a third container and a first, a second, and a third connector for sterile connection and liquid transfer between the cell culture vessel and the first container via the first connector, between the cell culture vessel and the second container via the second connector, and between the cell culture vessel and the third container via the third connector. In such instances, the first container may comprise a cell culture medium for transferring into the cell culture vessel, which comprises the cell culture, the second container may comprise a solution for transferring into the cell culture vessel, the solution comprising a nucleic acid for transducing the cells in the cell culture, and the third container may be a destination bag for receiving either the culture medium or the cells of the cell culture.

The system for non-parallel manufacturing of cells may further comprise one or more additional components involved in manufacturing cells. For example, systems disclosed herein may comprise a robotic device for moving the cell culture vessel and/or the connection interface, and/or for connecting the connector to the cell culture vessel and the container. Non-limiting examples of robotic devices include robots and robotic arms.

Any of the systems disclosed herein may comprise a controller, which schedules movement of the cell culture vessels in and out of the incubator, and/or in and out of the workstations. In some examples, the controller may comprise a memory circuit storing instructions and a processor circuit configured to execute the instructions. In some examples, the controller may comprise a memory circuit storing data from the manufacturing operations and sampling. In some instance, the controller is operated under a scheduling software, which can optimize performance based on various factors, including but not limited to, in-process data, pre-programmed instructions, and/or scheduling of sequential uses of the workstation. The scheduling software may function to manage processing of multiple cell cultures in the cell culture vessels. In some instances, the processing of the multiple cell cultures may be performed at a pre-determined manner. In other instances, the software may adjust such processing based on in-process data.

In some instances, the system allows for processing of the multiple cell cultures simultaneously. In other instances, the system allows for processing of the multiple cell cultures sequentially. In some examples, the system allows for processing of the multiple cell cultures in an independent manner depending upon factors such as in-process data.

### II. Connection Interfaces for Sterile Connection and Liquid Transfer

A connection interface for sterile connection and liquid transfer disclosed herein for use in the system for non-parallel manufacturing of cells may include one or more connectors and one or more sterilizable spaces, wherein the one or more connectors are operable to connect to two or more containers and/or a cell culture vessel in the sterilizable spaces. In some examples, the connection interface may further include one or more containers, which may be cell culture vessels, cell culture bags and/or containers for fluids, for example media or solutions of choice to be added. A container, as used herein, refers to any container suitable for holding a solution or suspension. A connector, as used herein, refers to an apparatus that is arranged to connect the containers in a sterilizable space.

A connection interface refers to an apparatus for connecting a container and a cell culture vessel via a connector in a sterilizable space (e.g., housing), and transferring a liquid between the container and the cell culture vessel thus connected. The connection interface may comprise one or more connectors and one or more sterilizable spaces, wherein the one or more connectors are operable to connect to one or more containers and/or a cell culture vessel in the sterilizable spaces. In some examples, the connection interface may further include one or more containers and/or a cell culture vessel. A container, as used herein, refers to any container suitable for holding a solution. A connector, as used herein, refers to an apparatus that is arranged to connect the container(s) and the cell culture vessel in a sterilizable space.

Any sterilizable space may be suitable for connecting the container(s) and the cell culture vessel via the connector. In some examples, a connection interface may include one or more sterilizable spaces. For example, the connection interface may include a housing that forms a sterilizable space for performing a sterile connection and liquid transfer. Alternatively, or in addition to, connectors optionally with an intermediate piece may form a sterilizable space. For example, the connection interface may include two or more pieces that form one or more sterilizable spaces for performing a sterile connection and liquid transfer. In some embodiments, the two or more pieces may be located in a housing that forms one or more sterilizable spaces.

In some examples, the sterilizable space may include a sterilizer and/or one or more ports operable to receive a source of the sterilizer for sterilization. Portions of the fluid conduit to the container(s), and the connectors with the optional intermediate piece may be sterilized in the sterilizable space in the housing of the connection interface using the sterilizer.

In some embodiments, the first connector or the second connector comprise a first piece and a second piece, wherein the first piece and the second piece form the first sterilizable space, a second sterilizable space, and/or a third sterilizable space; or the first connector or the second connector comprise a first piece and a second piece, the first piece and the second piece comprise one or more valves, one or more seals, and one or more ports.

In a preferred embodiment, the connection interface comprises a first connector and a second connector, wherein the first connector and the second connector define a sterilization chamber comprising a gap between the first connection surface and the second connection surface, wherein the gap is an enclosed space accessible through at least one opening, for example a port. The gap optionally comprises a sterilization agent. The first connector is fluidically coupled with a first container and the second connector is fluidically coupled with a second container and a liquid transfer device including pumps and valves allow for liquid transfer between the first container and the second container and/to avoid back contamination. In some instances, the gap between the first connection surface and the second connection surface is generated via partial coupling, wherein the gap is a closed space accessible through a port.

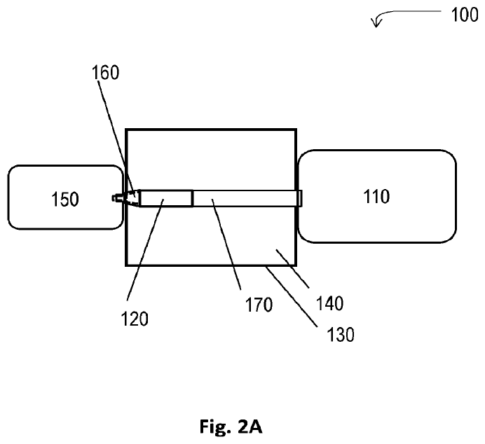

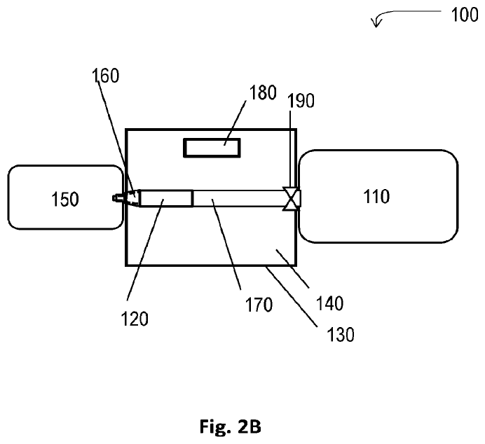

FIGS. 2A-2B are schematic depictions of an exemplary connection interface 100 including a first container 110, a connector 120, and a housing 130 forming a sterilizable space 140 for sterile connection and liquid transfer between a first container 110 and a second container 150 (for e.g., a cell culture vessel) via the connector 120, in accordance with some embodiments of the technology described herein. FIG. 2B shows the connector 120 attached to a connector 160 of the second container 150 and a fluid conduit 170 of the first container 110. FIG. 2A shows the connection interface of FIG. 2B further including a sterilizer 180, and a first container 110 that further includes a valve 190.

FIG. 2A shows a connection interface 100 that may include a connector 120 and connector 160 in a housing 130 that forms a sterilizable space 140 for sterile connection and liquid transfer between a first container 110 and a second container 150. The connectors may removably connected to the first container 110 and the second container 150, which may be a cell culture vessel. The connector 120 may be attached to the container 110 and the second container 150 such as a cell culture vessel in any suitable manner. For example, connector 120 is fluidly attached to a fluid conduit 170 of the first container 110 and to a connector 160 of the second container 150. In some examples, the connection interface 100 may include a first container 110, wherein the container may be a cell culture vessel. In one embodiment, connectors 120 and 160 mechanically interlock to form a fluid path. In another embodiment, connector 120 comprises a cannula and connector 160 comprises a septum. In some examples, the first container includes a solution for transferring into one of the cell culture vessels. In some instances, the solution is a culture medium and comprises one or more of: a viral particle or a nucleic acid that encodes a chimeric receptor.

Alternatively or in addition, the second container is one of the cell culture vessels. In some instances, the second container may comprise a destination bag for receiving either a culture medium or multiple cells from a cell culture. In an embodiment, the connection interface for sterile connection and liquid transfer comprises the first container including a solution for transferring into one of the cell culture vessels, wherein the solution is a culture medium and comprises one or more of: a viral particle or a nucleic acid that encodes a chimeric receptor. In other embodiments, the second container comprises a cell culture vessel. In some embodiments a solution in the first container is a culture medium for culturing cells grown in the second container.

FIG. 2B shows the connection interface that may include a valve or other suitable arrangement for controlling liquid flow and/or discouraging backflow. The valve may be a one-way valve for allowing fluid flow in a single direction or a bidirectional valve for allowing fluid flow in either direction. The valve may optionally be a pinch valve external to flexible tubing. The fluid conduit 170 of the first container 110 may include a valve 190 (e.g., a check valve) to control the flow of liquid and discourage backflow. The valve may be located in any suitable position, e.g., at a proximal end of the respective fluid conduit. It will be appreciated that other suitable arrangements may be utilized to encourage fluid flow in the desired direction. For example, the connection interface may include interlocked process controls. In another example, the connection interface may further include a pump operable to pump the contents between the first container and the second container, in either direction. In some embodiments, the connection interface further comprises a pump for liquid transfer between the second container and the first container, a second container, and/or the third container. For example, a pump may start just before opening a valve, creating positive or negative pressure to assure immediate flow of fluid in the desired direction.

The connection interface may include a sterilizer for sterilizing the sterilizable space and components placed within that space. Housing 130 may include a sterilizer 180 that sterilizes the sterilizable space 140 and components of the connection interface placed within the sterilizable space 140, including the connector 120 and portions of the first container 110 and second container 150. Sterilization may be performed before connecting the first container to second container via connectors 120 and 160 and/or after disconnecting the first container and the second container.

In some embodiments, the configuration may include a device for liquid transfer such a pump configured to transfer a liquid from the first container to the second container. Other examples for means to transfer a liquid from the first container to the second container are vacuum, a pressurizer and/or gravity. An interlock valve may be included to avoid back contamination between the first container and the second container. For example, a pump may be connected to the intermediate tubing, the fluid conduit of the container, and/or the fluid conduit of the cell culture vessel. Alternatively, or in addition to, the connection weld may use gravity to facilitate liquid transfer.

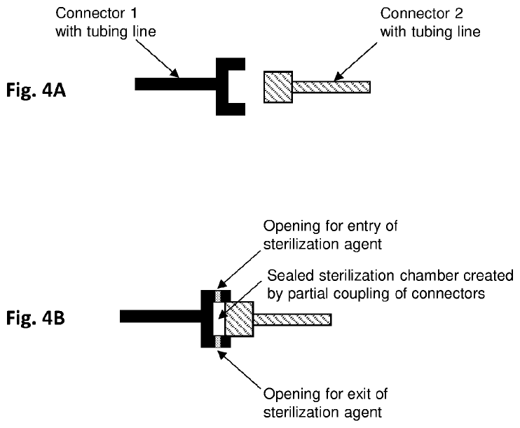

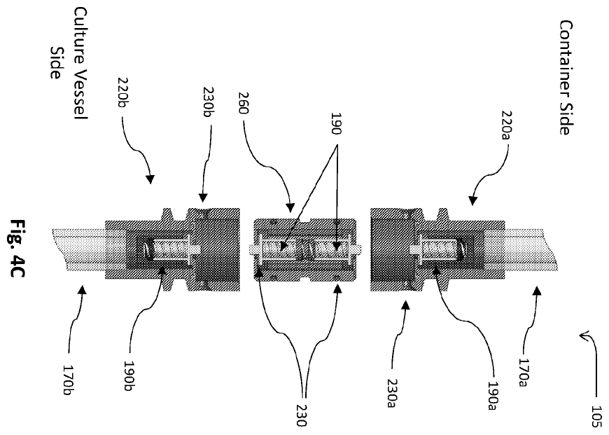

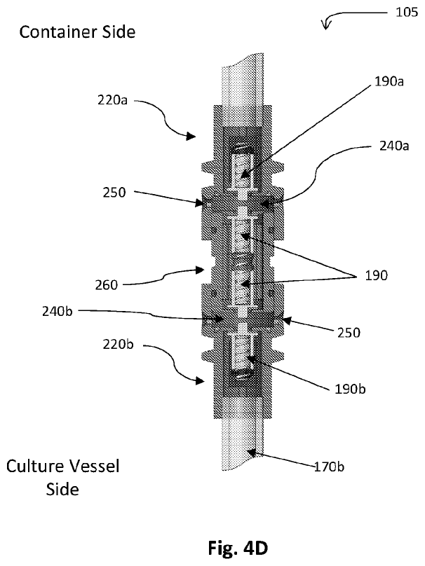

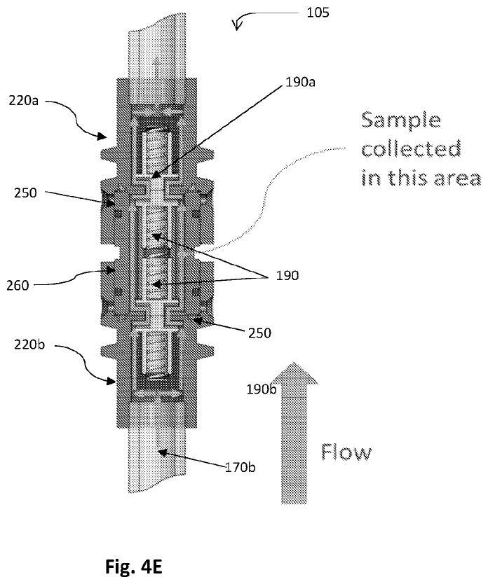

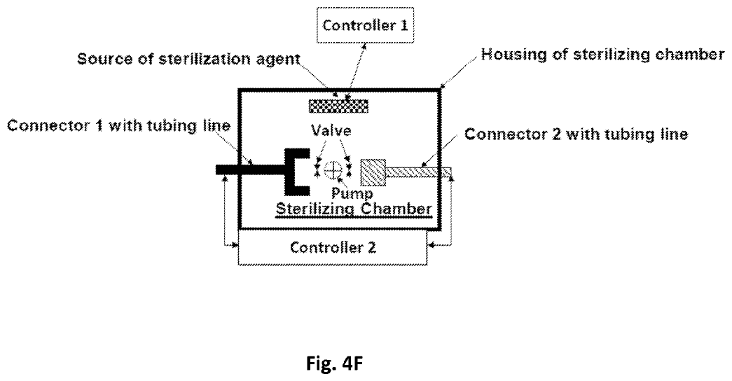

FIGS. 4A-4F are schematic depictions of exemplary connectors and connector interfaces, in accordance with some embodiments of the technology described herein.

FIG. 4A is a simplified version of an engineering schematic of designed fittings. Accordingly, the first connector (including a tubing line) and the second connector (including a tubing line) may be configured to form a sealed sterilization chamber by partially coupling the connectors. A first opening for entry of the sterilization agent and a second opening for exit of the sterilization agent are formed in the sealed sterilization chamber. Accordingly, a fluid sterilization agent may flow through the sealed sterilization chamber to effectively sterilize the connection interface. The fluid sterilization agent may be a gas, a liquid, or a hot vapor (e.g., water), and the like. In some embodiments, the sterilization agent comprises a fluid, a gas, or a vapor.

FIG. 4C shows the connection assembly 105 with the connector 220a attached to the fluid conduit 170a of the first container, connector 220b attached to the fluid conduit 170b of the second container, and an intermediate piece 260. In some embodiments, the connectors 220a and 220b may be arranged to form one or more sterilizable spaces. The intermediate piece 260 may removably connect to the connector 220a and the connector 220b. The connector 220a may include a valve 190a and a seal 230a, the intermediate piece 260 may include two valves 190 and two seals 230, and the connector 220b may include a valve 190b and a seal 230b. The valves 190 may be operable to control the flow of fluid through the connector. The seals 230 may be operable to provide fluid and air tight seals between the connector 220a and the intermediate piece 210 and between the connector 220b and the intermediate piece 260. In another example, the two valves 190 is eliminated and the intermediate piece 260 is connected to the fluid conduit 170b, creating only a single sterilizable space.

FIG. 4D shows additional spaces 240a and 240b for sterile connection and liquid transfer formed from attaching each end of the intermediate piece 260 to connectors 220a and 220b. The additional space 240a and 240b may be sterilized via ports 250a and 250b (hereinafter, collectively referred to as “ports 250”) in the connectors. In some embodiments, one or more sterilizable spaces 240a and 240b are formed by coupling each end of the intermediate piece 260 to the first and second connector 220a and 220b. The sterilizable space 240a and 240b may be sterilized via ports 250 in the first and second connectors 220a and 220b. For example, the ports 250 may be operable to receive a source of steam for sterilizing the sterilizable space 240a between the connector 220a and the intermediate piece 260 and the sterilizable space 240b between the connector 220b and the intermediate piece 260. The sterilization spaces formed by partial compression of the connectors 220a and 220b are maintained by spring-held valves 190, 190a and 190b at the connection surface, obstructing the sterilizing agent from the fluid flow path (170a and 170b).

FIG. 4E shows the connectors (e.g., 220a, an example of connector 120; 220b, an example of connector 160, and intermediate piece (e.g., 260) described in FIG. 4C—D), fully engaged to allow fluid transfer. The opposing pins of spring valves, e.g. 190, 190a and 190b, are pushed open by compression of the connectors 220a and 220b to unseal the fluid flow path. This figure notes that the seals 230a, 230b of the intermediate piece 260, when broken, will contain fluid between the seals and therefore can be used for sample removal. In one embodiment, valves 190 and 190a are configured to comprise opposing pins which, upon compression of the connector 220a and the intermediate piece 260, meet each other and, against the springs within the valves, open the flow path.

FIG. 4F shows a connection interface, according to some embodiments. The connection interface includes a sterilizable chamber within a housing. The sterilizable chamber is configured to receive a first connector fluidically coupled to a for e.g., first container and a second connector fluidically coupled to a for e.g., second container. In some embodiments, a sterilization agent in the first sterilizable chamber is activated to sterilize the first connector and the second connector. In some embodiments, the connection interface may also include a pump configured to transfer a liquid from the first container to the second container. In some embodiments, the connection interface may include an interlock valve configured to avoid back contamination between the first container and the second container. In some embodiments, the pump may be a peristaltic pump, including one or more interlocking pinch valves between the first container and the second container to prevent backflow between the first container and the second container. In some embodiments the pump and/or pinch valves may be located inside the sterilizable space. In other embodiments the pump and/or pinch valves may be located outside the sterilizable space.

In some embodiments, the connection interface may include a first controller configured to activate the sterilization agent over the first connector and the second connector. In some embodiments, the connection interface may include a second controller configured to load the first connector and the second connector into the sterilizable chamber and/or to remove the first connector or the second connector from the sterilizable chamber after liquid transfer between the first container and the second container.

A connector may be removable and/or disposable and/or reusable. The connector may be removed (or disconnected) from a container, e.g., a cell culture vessel, after performing a sterile connection and liquid transfer. Any suitable manner may be used to remove the connector from the connection interface, e.g., by ejecting the connector from the connection interface. A connector may be disposable such that a new connector may be used for each sterile connection and liquid transfer between a first container and a second container. A connector may be reusable such that it may be used in multiple sterile connections and liquid transfers. In such instances, the connector may be sterilized prior to each use. In some instances, there may be multiple connectors.

In some embodiments, the first connector, the second connector, and/or the third connector is removable, disposable, reusable, or a combination thereof. In some embodiments, the first connector, the second connector, and/or the third connector is ejectable (e.g., a force (e.g., from a tensed spring) separates the first, second and/or third connector upon an external trigger (e.g., mechanical, electrical or magnetic pulse) from the connection interface. In some embodiments, the first container, the second container, and/or the third container comprises a fluid conduit, and the first connector, the second connector, and/or the third connector is arranged to be attached to the fluid conduit. In other embodiments, the second container comprises a septum, and the first connector, the second connector, and/or the third connector is arranged to be attached to the septum.

A connector may include one or more parts, e.g., one or more cannulas and/or one or more septae, and/or one or more mechanical fittings and/or one or more pieces of tubing. For example, when the connector includes a single piece of tubing, one end of the tubing is arranged to be welded to a fluid conduit of a container and the other end of the tubing is arranged to be welded to a fluid conduit of a cell culture vessel. In another example, when the connector includes multiple pieces of tubing, an intermediate portion of tubing may be used to connect two or more pieces of tubing. In some instances, multiple connectors are connected. In exemplary embodiments, the first connector, and/or the second connector each comprise a first piece and a second piece, one end of the first piece being arranged to be attached to the first container, a second container, and/or the third container and one end of the second piece being arranged to be attached to the second container. In some instances, the first connector or the second connector comprise a first piece and a second piece. The first connector or the second connector may further comprise an intermediate piece having a first end and a second end, which are arranged to be attached to a second end of the first piece and/or a second end of the second piece. In some embodiments, the first connector, the second connector, and/or the third connector comprises a septum and/or a cannula.

An optional intermediate piece may be removable and/or disposable. Alternatively, or in addition to, the intermediate piece may be configured for liquid sampling through one or more ports. In some embodiments, the first connector or the second connector comprise a first piece and a second piece, further comprising an intermediate piece having a first end and a second end, which are arranged to be attached to a second end of the first piece and/or a second end of the second piece. In some embodiments, the first connector or the second connector each comprise a first piece and a second piece. The first container and the second container may comprise a fluid conduit. The first piece can be arranged to be attached to the fluid conduit. The second container can comprise a septum, and the second piece is arranged to be attached to the septum. In some examples, the first connector or the second connector may comprise a first piece and a second piece. The first piece and the second piece can form the first sterilizable space, a second sterilizable space, and/or a third sterilizable space.

In any of the systems disclosed herein, the sterilizer agent comprises an energy source selected from the group consisting of UV light, e-beams, gamma rays, heat, and steam. In preferred embodiments, the sterilizer agent comprises a fluid selected from a gas, or a vapor.

A connector may include one or more features useful for sterile connection and liquid transfer. For example, a connector may include one or more valves to control the flow of liquid. In another example, a connector may include one or more seals to prevent leakage. In yet another example, a connector may include one or more ports to allow access to a sterilizing agent, e.g., steam. In some embodiments, the first connector or the second connector can comprise a first piece and a second piece. The first piece and the second piece may comprise: one or more valves, one or more seals, and one or more ports.

A container for use in a connection interface disclosed herein may include a fluid conduit and/or opening for transferring liquid between the container and a second container. For example, the container may include a fluid conduit, which may be attached to a connector when performing a sterile liquid transfer. Alternatively, or in addition to, the container may include an opening including a septum for transferring liquid under sterile conditions.

Liquid may be transferred between the first container and the second container in either direction. In some embodiments, liquid may be transferred from the first container to the second container. In some embodiments, liquid may be transferred to the first container from the second container. The first container may be empty to receive the contents of the second container or the first container may include a solution to be transferred into the second container. In other embodiments, the first connector or the second connector each comprise a first piece and a second piece, the first container and the second container comprise a fluid conduit, and the first piece is arranged to be attached to the fluid conduit, and the second container comprises a septum, and the second piece is arranged to be attached to the septum.

A non-limiting example of a solution to be transferred from the container to the cell culture vessel is a culture medium for culturing cells in the cell culture vessel. Alternatively, or in addition, the solution includes a nucleic acid for transducing cells grown in the cell culture vessel. Such nucleic acids may be delivered into cells using conventional technologies, e.g., transduction using reagents such as liposomes or viral transduction (e.g., retroviral transduction such as lentiviral transduction). When the connection interface is being used to manufacture cells expressing a chimeric antigen receptor (CAR), the solution may include a nucleic acid encoding the CAR. In some embodiments, the solution in the first container comprises a nucleic acid or a viral particle comprising such for transducing cells grown in the second container, and wherein the nucleic acid encodes a chimeric receptor. In some embodiments, the second container comprises a destination bag for receiving either culture medium or multiple cells from a cell culture.

In some embodiments, the second container comprises a cell culture and the first container is a destination bag for receiving either a culture medium or multiple cells in the cell culture. Alternatively, the first container comprises a cell culture medium or a viral vector for transferring into the second container, which comprises a first cell culture. The connection interface may further comprise a third container including a second cell culture in one of the cell culture vessels configured to receive the cell culture medium or the viral vector from the first container. In some embodiments, the first container comprises a cell culture medium or a viral vector for transferring into the second container, which comprises a first cell culture, wherein the connection interface further comprises a third container including a second cell culture in one of the cell culture vessels configured to receive the cell culture medium or the viral vector from the first container.

Any suitable number of containers and/or connectors may be included in a connection interface disclosed herein. In some embodiments, a connection interface may include one or more containers and two or more connectors for sterile connection and liquid transfer between a cell culture vessel and the one or more containers via the one or more connectors. In such instances, the one or more containers and the cell culture vessel may be connected via the two or more connectors in the same sterilizable space or in different sterilizable spaces.

In some embodiments, the connection interface disclosed herein may comprise multiple containers, one being the source container and the others being the destination containers. A liquid (e.g., cell culture medium) can be transferred between contains in a one-to-many manner, e.g., from the source container to each of the destination containers (e.g., containing cells) in a sequential manner via serial connections and disconnections.

In some embodiments, the connection interface disclosed herein may comprise multiple containers, one being the destination container (e.g., containing cells) and the others being the source containers (containing culture medium, viral vectors, growth factors, etc.). A liquid can be transferred in a many-to-one manner between containers, e.g., from each of the multiple source containers to the destination container in a sequential manner via serial connections and disconnections.

In some embodiments, a connection interface may include a first and a second container and a first and a second connector for sterile connection and liquid transfer between the cell culture vessel and the first container via the first connector, and between the cell culture vessel and the second container via the second connector. In such instances, the first container may include a cell culture medium for transferring into the cell culture vessel, which includes the cell culture, and the second container may be a destination bag for receiving either the culture medium or the cells of the cell culture.

In some embodiments, a connection interface may include a first, a second, and a third container and a first, a second, and a third connector for sterile connection and liquid transfer. This setup can be used for liquid transfer between a cell culture vessel and the first container (e.g., containing media) via the first connector, between the cell culture vessel and the second container (e.g., containing waste) via the second connector. In a further embodiment this setup can be easily extended with a fourth container (e.g., containing a suspension with a non-viral or viral vector for cell transduction) and a fourth connector between the cell culture vessel and the fourth container, or can be extended with multiple further pairs of containers and connectors for e.g., adding solutions comprising compounds for cell manipulation. Similarly, in some embodiments, a connection interface may include multiple pairs of containers being cell culture vessels with different cell cultures and connectors, and one or more pairs of containers and connectors for media, and/or solutions/suspensions for cell manipulation.

A connection interface disclosed herein may further include a housing. The housing may surround and/or contain the connector and one or more sterilizable spaces. In some embodiments, the housing forms a sterilizable space. A housing may be any suitable shape or size, and any suitable material.

A housing may surround one or more connectors or portions thereof. For example, the housing may surround a first connector and second connector. In another example, the housing may surround a first connector, a second connector, and a third connector.

A housing may contain one or more connectors or portions thereof. For example, the housing may contain a first connector and second connector. In another example, the housing may contain a first connector, a second connector, and a third connector.

A housing may surround one or more containers or portions thereof. Alternatively, or in addition to, the housing may surround one or more cell culture vessels or portions thereof. For example, the housing may surround a fluid conduit of a container and/or a fluid conduit of a cell culture vessel. In another example, the housing may surround a fluid conduit of a container and a septum of a cell culture vessel.

A housing may contain one or more containers or portions thereof. Alternatively, or in addition to, the housing may contain one or more cell culture vessels or portions thereof. For example, the housing may contain a fluid conduit of a container and/or a fluid conduit of a cell culture vessel. In another example, the housing may contain a fluid conduit of a container and a septum of a cell culture vessel.

A connection interface disclosed herein may further include a device that facilitates sterile connection and/or liquid transfer. For example, the connection interface may include a device for sterile connection such as a sterilizer. The sterilizer may include an energy source that directs energy towards the sterilizable space and components placed within that space. The energy source preferably is UV light, e-beams, gamma rays, heat and/or steam, preferably heat and/or steam. Alternatively or additionally, the sterilizer may be a sterilizer agent, preferably a fluid selected from a gas (e.g. ozone), a sterilizing chemical (e.g. ethanol) or a vapor.

In another example, the connection interface may include a device for liquid transfer such as a pump, a vacuum, or a pressurizer. For example, a pump may be connected to the connector, the fluid conduit of the container, and/or the fluid conduit of the cell culture vessel. Alternatively, or in addition to, the connection interface may use gravity to facilitate liquid transfer. Any of the devices for sterile connection and/or liquid transfer may be located in the housing of the connection interface.

### III. Methods for Non-Parallel Processing of Multiple Cell Cultures

Also provided herein are methods for non-parallel processing of multiple cell cultures, each of which may be in a cell culture vessel. In other aspects, the present disclosure features a method for non-parallel processing of multiple cell cultures, the method comprising: (i) providing any of the systems for manufacturing cells as disclosed herein, wherein the system comprises multiple cell culture vessels, each of which comprises a cell culture; and (ii) performing manufacturing operations on one or more of the cell cultures in the multiple cell culture vessels. In some instances, the manufacturing operations on the multiple cell cultures in the multiple cell culture vessels are not parallel. In some embodiments, the manufacturing operations comprise centrifugation, mixing, media removal, media addition, feed addition, vector addition, sampling, buffer addition, buffer removal, or a combination thereof.

As used herein, non-parallel processing refers to manufacturing multiple cell cultures such that manufacturing steps (e.g., transferring liquid, centrifuging, or incubating) may be carried out on the multiple cell cultures at different times. For example, one of the cell cultures may be centrifuged while another cell culture is involved in a liquid transfer. As such, multiple cell culture vessels may be moved independently during non-parallel processing. Alternatively or in addition, the manufacturing operations comprise sterile connection and liquid transfer between at least one of the cell culture vessels and a bioprocess container. In some examples, the bioprocess container is a media bag, a buffer bag, a sample container, or a waste container. In some embodiments, the method comprises performing the same manufacturing operation on multiple cell cultures in the multiple cell culture vessels simultaneously or sequentially. In some embodiments, the method comprises performing the same manufacturing operation on multiple but not all cell cultures in the multiple cell culture vessels simultaneously or sequentially. In other embodiments, the method comprises performing different manufacturing operations on different cell cultures in the multiple cell culture vessels simultaneously or sequentially.

Further, in an exemplary embodiment, the manufacturing operations of multiple cell cultures comprise a connection interface for sterile connection and liquid transfer between at least one of the cell culture vessels and a bioprocess container. Also provided herein are methods for sterile connection via tube welding for multiple sequential weld connections to a single source and/or destination container, e.g., to enable cell therapy manufacturing and automation of cell therapy manufacturing operations. Methods disclosed herein involve sterile connection and liquid transfer between any source vessel and destination vessel wherein operates automated, manually, or a combination thereof.

In such instances, methods disclosed herein may be performed to transfer the culture media from the source vessel to the cell culture in the destination vessel. One embodiment comprises a method wherein the sterile liquid transfer of the connection interface further comprises (a) placing a first tube and a second tube into a coupling mount, wherein the first tube is connected to a first container and the second tube is connected to a second container; (b) coupling the first tube and the second tube to form a first sterile fluidical connection between the first container and the second container; (c) transferring a liquid between the first container and the second container via the first sterile fluidical connection; (d) sealing and cutting the first fluidical connection between the first container and the second container to disconnect the first sterile fluidical connection.

Further, in an embodiment, the method is further applied for sterile liquid transfer with a third container comprising the further steps (e) placing a third tube into the coupling mount, wherein the third tube is connected to a third container; (f) welding the first or second tube and the third tube to form a second sterile fluidical connection between the first container and the third container or between the second container and the third container; (g) transferring a liquid between the first container and the third container or between the second container and the third container via the second sterile fluidical connection; and (h) sealing the second fluidical connection between the connected first and third tubes or between the connected second and third tubes to disconnect the second sterile fluidical connection. Non limiting examples of a coupling mount includes welding, soldering, valve or port. In some embodiments, steps (e) and (f) comprise welding, on one side of an existing weld, a selected length of a tubing, and adding new welds to the added tubing until the selected length of tubing is used up. Steps (a) to (d) or steps (e) to (h) are optionally repeated.

In some embodiments, coupling the first tube to the second tube and/or the first or second tube to the third tube comprises coupling a fresh portion of a tube of a pre-selected length in-between the first tube and the second tube, or in-between the first or second tube and the third tube, thereby connecting the first tube and second tube, or connecting the first or second tube to the third tube. In exemplary embodiments, coupling the first tube and the second tube, and/or coupling the first or second tube and the third tube comprises (i) forming two separate sterile connections in the first tube and the second tube or in the first or second tube and the third tube with a heated welder blade, a laser or a cold blade combined with a heating element, a heated welder blade, and/or (ii) welding, on one side of an existing weld, a selected length of a tubing, and adding a new weld to the opposite side of the existing weld until the selected length of tubing is used up.

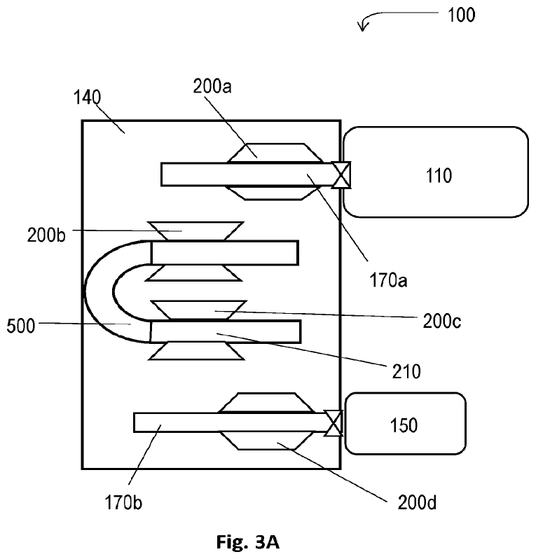

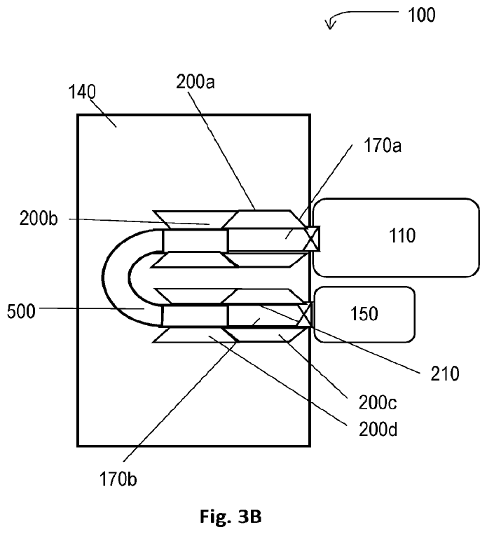

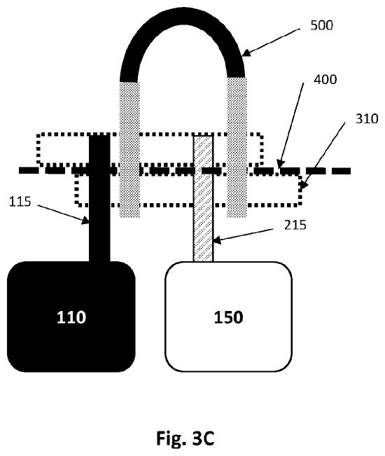

FIG. 3A, is a schematic depiction of a connection interface 100, which may comprise multiple weld heads 200a-d into which the fluid conduit 170a of the first container 110, the fluid conduit 170b of the second container 150 such as a cell culture vessel, and a connector such as tubing 210 having an intermediate portion 520, may be inserted. The intermediate portion may be a new piece of tubing or it may be formed from a longer piece of tubing that is sealed into the intermediate portion. The intermediate portion of tubing may be removable and/or disposable. Alternatively, or in addition to, the intermediate portion of tubing may be configured for liquid sampling. FIG. 3A shows the fluid conduit 170a of the first container 110, the fluid conduit 170b of the second container 150 such as a cell culture vessel, the tube 210 with the weld heads, prior to being welded together. Welds may be performed via the weld heads to connect the fluid conduit 170a of the first container 110 and the fluid conduit 170b of the second container 150 such as a cell culture vessel via the destination tube 215. For example, the fluid conduits may be connected via tube portion 500 (the weld heads have been omitted) as shown in FIG. 3B. Once a connection has been established between the container and the cell culture vessel, liquid transfer may be performed. Non-limiting examples of liquid transfer include taking samples from the cell culture vessel, removing waste from the cell culture vessel, and transferring media and/or solution(s) from the container to the cell culture vessel. FIG. 3C shows two welds with one weld head and one blade for connecting two containers. It shows the alignment of the cut ends of source container tubing 115 with one end of the intermediate/spool portion 500 and cut ends of the destination container tubing 215 with the other end of the intermediate/spool portion 500 after movement of the weld mounts 310 still separated by the heated welder blades 400; cut ends have been discarded.

A sterile connection and liquid transfer may be performed before and/or after processing of the cell culture in the cell culture vessel. For example, the cell culture vessel including the cell culture may be centrifuged and/or mixed prior to performing a sterile connection and liquid transfer. In another example, the cell culture vessel including the cell culture may be centrifuged and/or mixed after performing a sterile connection and liquid transfer. In yet another example, the cell culture vessel including the cell culture may be centrifuged and/or mixed both before and/or after performing a sterile connection and liquid transfer.

Methods disclosed herein encompass any moving of the cell culture vessel and the connection weld such that a sterile connection and liquid transfer may be performed. Accordingly, the moving step may involve moving the cell culture vessel to the connection weld or moving the connection weld to the cell culture vessel. Alternatively, or in addition to, the moving step may involve moving both the cell culture vessel and the connection weld.

Liquid transfer may be achieved using any suitable method for transferring liquids, e.g., transfer via gravity or a device such as a pump, vacuum, or pressurizer.

Connection welds and methods for sterile connection and liquid transfer described herein can be used for manufacturing cells, e.g., manufacturing immune cells expressing a chimeric antigen receptor. Manufacturing cells may include culturing cells, expanding cells, or transducing cells. Manufacturing cells may involve any number of connection welds used to perform any number of sterile connections and liquid transfers.

In some embodiments, multiple source containers may be connected sequentially to a single destination container (e.g., for adding/removing media and solutions to a single cell culture vessel). In some embodiments, multiple destination containers may be connected sequentially to a single source container (e.g., for adding media to multiple cell culture vessels). In some embodiment, multiple source containers may be connected sequentially to multiple destination containers (e.g., for adding/removing media and solutions to multiple cell culture vessels).

Such methods may use multiple containers to transfer cells and/or reagents into a cell culture vessel for manufacturing cells, e.g., for transducing cells. For example, the first container may include a cell culture medium for culturing cells, the second container may include a solution including a nucleic acid for transducing the cells, and the third container may be a destination bag for receiving either the cell culture medium or the cells.

Methods disclosed herein may also involve collecting the cells. For example, methods disclosed herein may result in collection of the cells in a container such as a destination bag. As such, methods disclosed herein may further include centrifuging a cell culture to obtain the collection of cells. Cells may be collected at any point during the manufacturing process, e.g., when transferring cells to a larger cell culture vessel during cell expansion or when harvesting cells for downstream processing or therapeutic use. Accordingly, cells may be collected in any one of the containers (e.g., the first, second, or third containers) used when performing multiple sterile connections and liquid transfers.

Methods disclosed herein may involve any one of the systems for non-parallel manufacturing of cells disclosed herein.

A manufacturing step encompasses any procedure, process, and/or practice related to manufacturing cells. Manufacturing steps include, but are not limited, to one or more of incubating cells, analyzing cells, separating cells, processing cells, aliquoting cells and/or reagents, addition or removal of media or buffer, addition of vector or reagents as growth factors, performing a sterile connection and liquid transfer, and transferring cells and/or liquids. A manufacturing step may involve a workstation, e.g., separating cells in a cell separation workstation such as a centrifuge).

Methods disclosed herein may involve any number of manufacturing steps. For example, methods may comprise performing one or more manufacturing steps, e.g., performing a first, a second, and a third manufacturing step. Any number of manufacturing steps may be performed on a cell culture.

Methods disclosed herein may involve any number and/or any type of cell cultures. Accordingly, methods disclosed herein may be used for manufacturing various quantities and types of cells.

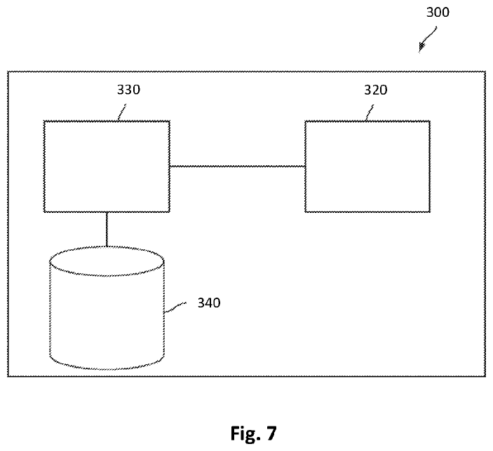

An illustrative implementation of a computer system 300 that may be used in connection with some embodiments of the technology disclosed herein is shown in FIG. 7. The computer system 300 may include one or more processors 330 (e.g., processing circuits) and one or more computer-readable storage media (i.e., tangible, non-transitory computer-readable media), e.g., volatile storage 320 (e.g., memory) and one or more non-volatile storage media 340, which may be formed of any suitable non-volatile data storage media. The processor(s) 330 may control writing data to and reading data from the volatile storage 320 and/or the non-volatile storage device 340 in any suitable manner, as aspects of the present invention are not limited in this respect. To perform any of the functionality described herein, processor(s) 330 may execute one or more instructions stored in one or more computer-readable storage media (e.g., volatile storage 320), which may serve as tangible, non-transitory computer-readable media storing instructions for execution by the processor 330.

Embodiments of the present invention can be implemented in any of numerous ways. For example, the embodiments may be implemented using hardware, software or a combination thereof. When implemented in software, the software code (e.g., instructions) can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above. In some embodiments, the control of unit operations may be performed via an integrated 3^{rd} party software or control on a particular device, while a global system (e.g., SCADA) may be provided for supervisory control, data acquisition, and/or scheduling.

In this respect, it should be appreciated that one implementation of embodiments of the present invention comprises at least one computer-readable storage medium (i.e., at least one tangible, non-transitory computer-readable medium, e.g., a computer memory, a floppy disk, a compact disk, a magnetic tape, or other tangible, non-transitory computer-readable medium) encoded with a computer program (i.e., a plurality of instructions), which, when executed on one or more processors, performs above-discussed functions of embodiments of the present invention. The computer-readable storage medium can be transportable such that the program stored thereon can be loaded onto any computer resource to implement aspects of the present invention discussed herein. In addition, it should be appreciated that the reference to a computer program which, when executed, performs above-discussed functions, is not limited to an application program running on a host computer. Rather, the term “computer program” is used herein in a generic sense to reference any type of computer code (e.g., software or microcode) that can be employed to program one or more processors to implement above-discussed aspects of the present invention.

In some embodiments, the multiple cell culture processing method disclosed herein comprise sterile connection and liquid transfer between a cell culture vessel and a bioprocess container as disclosed herein. FIGS. 4A-4F are schematic depictions of an exemplary process for sterile connection and liquid transfer, in accordance with some embodiments of the technology described herein. The process may include an exemplary method for sterile connection and liquid transfer between a source vessel (e.g., a container) and a destination vessel (e.g., a cell culture vessel) using a connection interface disclosed herein.

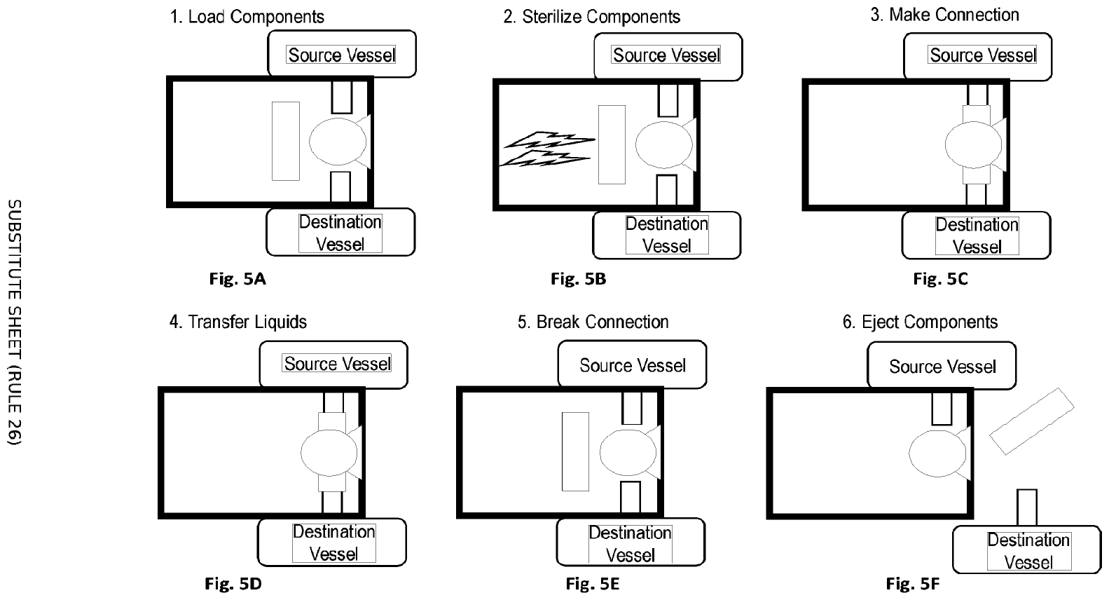

FIG. 5A illustrates the connection interface being loaded with the source vessel, the destination vessel, and a connector (rectangle). In this illustrative example, the connection interface includes a pump (circle) to transfer liquid from the source vessel to the destination vessel. FIG. 5B illustrates portions of the vessels (e.g., fluid conduits) and the connector that are then sterilized in the sterilizable space in the housing of the connection interface. FIG. 5C illustrates that the source vessel is then connected to the destination vessel via the connector. FIG. 5D shows liquid transfer from the source vessel to the destination vessel. FIG. 5E illustrates that the source vessel and the destination vessel are disconnected from the connector, after the desired amount of liquid is transferred. FIG. 5F illustrates that the connector and destination vessel are then ejected from the connection interface.

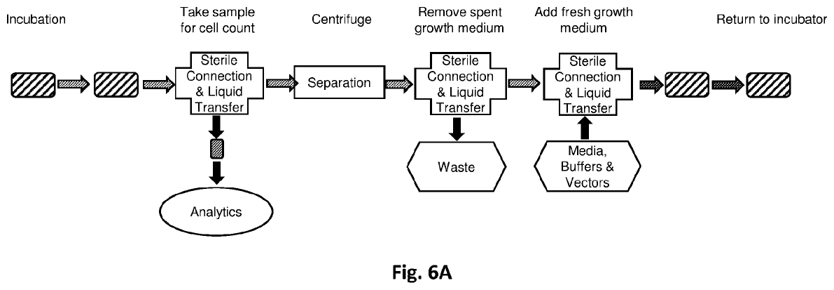

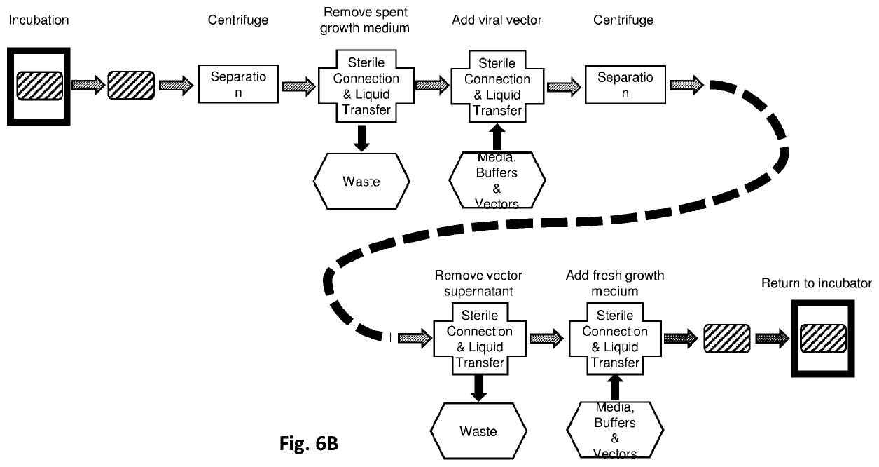

FIGS. 6A-B are schematic depictions of an exemplary process for manufacturing cells, in accordance with some embodiments of the technology described herein.

FIG. 6A is a schematic depiction of an exemplary process for expanding a cell culture. A connection interface may be used to perform various liquid transfers involved in the cell expansion process including taking a sample of the cells for analysis, removing spent growth medium, and adding fresh medium. A device capable of performing serial sterile connection and liquid transfer operations is shown as the blue plus in the workflow of a typical cell culture manufacturing operation of cell growth medium addition. External steps may be performed manually, or containers may be transferred by robotic arms or similar automated transfer devices. Automated loading of the connectors, sterilization, connection, liquid transfer, and disconnection of containers are performed by the sterile connection and liquid transfer device. In this example, the sterile connection and liquid transfer device is used to first to remove medium (from the cell culture source bag to a destination waste medium bag) then to add liquid from the cell culture medium source bag to destination cell culture bag). Note that the waste medium destination bag and cell culture medium source bag may be serially connected to many cell culture bags (acting first as source bags, then as destination bags.

FIG. 6B is a schematic depiction of an exemplary process for transducing a cell culture. A connection interface may be used to perform various liquid transfers involved in the cell transduction process including adding viral vector, removing vector supernatant, removing spent growth medium, and adding fresh medium. A device capable of performing serial sterile connection and liquid transfer operations is shown as the blue plus in the workflow of a typical CAR-T cell therapy manufacturing operation of viral vector transduction. External steps may be performed manually, or containers may be transferred by robotic arms or similar automated transfer devices. Automated loading of the connectors, sterilization, connection, liquid transfer, and disconnection of the containers are performed by the sterile connection and liquid transfer device. In this example, the sterile connection and liquid transfer device is used to first to remove medium (from the cell culture source bag to a destination waste medium bag) then to add liquid from the viral vector source bag to destination cell culture bag).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

It should be appreciated that various embodiments of the present invention may be formed with one or more of the above-described features. The above aspects and features of the invention may be employed in any suitable combination as the present invention is not limited in this respect. It should be appreciated that the drawings illustrate various components and features which may be incorporated into various embodiments of the present invention. For simplification, some of the drawings may illustrate more than one optional feature or component. However, the present invention is not limited to the specific embodiments disclosed in the drawings. It should be recognized that the present invention encompasses embodiments which may include only a portion of the components illustrated in any one drawing figure, and/or may also encompass embodiments combining components illustrated in different figures.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A system for non-parallel random access manufacturing of cells, wherein the system comprises:
(a) one or more incubator arranged to house a plurality of cell culture vessels, wherein each cell culture vessel is configured for moving in and out of the incubator(s) independently;
(b) one or more workstation configured to host each of the cell culture vessels to perform one or more manufacturing operations; and
(c) a transfer device for moving the cell culture vessels between two incubators, between the incubator and the workstation, or between two workstations;
wherein the transfer device of (c) operates automated, manually, or a combination thereof.

2. The system of claim 1, wherein the one or more manufacturing operations of (b) comprise centrifugation, mixing, media removal, media addition, feed addition, vector addition, sampling, buffer addition, buffer removal, or a combination thereof.

3. The system of claim 1, wherein the one or more workstations of (b) are configured for sterile connection and liquid transfer between the cell culture vessels and one or more bioprocess containers, wherein the one or more bioprocess containers comprise media bags, buffer bags, sample containers, waste containers, or a combination thereof.

4. The system of claim 1, wherein the cell culture vessel comprises:
(A) an inner container comprising a pocket, wherein the pocket defines a volume within which a cell culture is maintained during manufacturing of cells; and
(B) an outer shell configured to receive and support the inner container, wherein the outer shell includes a shell top and a shell bottom that cooperate with one another to form a chamber within which the inner container is disposed, optionally, encapsulated.

5. The system of claim 4, wherein the volume of the pocket is arranged to maintain the cell culture is adjustable optionally, wherein the outer shell comprises the at least one clamp and the volume of the pocket is adjustable via the clamp, which optionally is a sliding clamp

6. The system of claim 1, further comprising a controller, the controller includes:
(I) a processor;
(II) a memory storing manufacturing operations, sampling and instructions that, when executed by the processor, cause the processor to:
(a) schedule movements of the cell culture vessels between the incubator(s) and the workstations, wherein the movements are configured to execute automatically.

7. The system of claim 6, wherein the controller schedules the movements based on threshold of analytical in-process data, which optionally comprising: cell count, cell viability, level of transduction, growth medium properties, contaminants, or a combination thereof.

8. The system of claim 1, wherein one or more of the cell culture vessels of the plurality of cell culture vessels host a cell culture, and wherein the one or more manufacturing operations on the cell cultures in the plurality of cell culture vessels are performed simultaneously.

9. The system of claim 6, wherein the processor is further configured to execute one or more of the following:
(i) manage a plurality of cell cultures simultaneously; and
(ii) create a custom schedule for the cell culture in each of the cell culture vessels to manage process performance.

10. The system of claim 9, wherein the creating the custom schedule for the cell culture is based on pre-programmed instructions, in-process data, scheduling of sequential use of the workstations, or a combination thereof.

11. The system of claim 3, wherein one or more of the workstations comprise a connection interface for sterile connection and liquid transfer.

12. The system of claim 11, wherein the connection interface comprises:
a first connector;
a second connector;
wherein the first connector and the second connector define a sterilization chamber comprising a gap between the first connection surface and the second connection surface;
preferably, wherein the gap is an enclosed space accessible through at least one opening, preferably optionally a port;
wherein the gap optionally comprises a sterilization agent;
wherein the first connector is fluidically coupled with a first container and the second connector is fluidically coupled with a second container; and
(iii) a first sterilizable space, which optionally comprises a sterilization agent, wherein the first connector is fluidically coupled with a first container and the second connector is fluidically coupled with a second container in the first sterilizable space; and
(iv) a liquid transfer device including one or more pump or more valve, configured to facilitate liquid transfer between the first container and the second container to avoid back contamination.

13. The system of claim 2, wherein transferring a liquid between the first container and the second container comprises:
(i) interlocking one or more (pinch) valves and/or a (peristaltic) pumps;
(ii) slightly rotating the peristaltic pump to create a positive or a negative pressure in one of the first tube or the second tube prior to releasing the one or more pinch valves to cause a positive or a negative pressure in one of the first tube or the second tube;
(iii) rotating a peristaltic pump between two interlocking valves prior to activating the interlocking valves; and/or
(iv) pumping the liquid from the first container to the second container or pumping the liquid from the first or second container to the third container.

14. The system of claim 13, wherein the first container includes a solution for transferring into one of the cell culture vessels, wherein the solution is a culture medium and comprises one or more of: a viral particle or a nucleic acid that encodes a chimeric receptor.

15. The system of claim 14, wherein the second container is one of the cell culture vessels.

16. The system of claim 14, wherein a solution in the first container is one of (1) a culture medium for culturing cells grown in the second container, and (b) a solution in the first container comprises a nucleic acid or a viral particle comprising such for transducing cells grown in the second container, and wherein the nucleic acid encodes a chimeric receptor.

17. (canceled)

18. The system of claim 12, wherein the second container comprises a cell culture and the first container is a destination bag for receiving either a culture medium or multiple cells in the cell culture.

19. The system of claim 12, wherein the first container comprises a cell culture medium or a viral vector for transferring into the second container, which comprises a first cell culture, wherein the connection interface further comprises a third container including a second cell culture in one of the cell culture vessels configured to receive the cell culture medium or the viral vector from the first container.

20. The system of claim 12, wherein the second container comprises a destination bag for receiving either a culture medium or multiple cells from a cell culture.

21. The system of claim 12, wherein the sterilizer agent comprises one of (a) an energy source selected from the group consisting of UV light, e-beams, gamma rays, heat, and steam, and (b) a fluid selected from a gas, or a vapor.

22. (canceled)

23. The system of claim 12, wherein the connection interface further comprises a pump for liquid transfer between the second container and the first container, a second container, and/or the third container.

24. The system of claim 12, wherein the first connector, the second connector, and/or the third connector is removable, disposable, reusable, or a combination thereof.

25. The system of claim 12, wherein the first connector, the second connector, and/or the third connector comprises a septum and/or a cannula.

26. The system of claim 12, wherein the first connector, the second connector, and/or the third connector is ejectable from the connection interface.

27. The system of claim 12, wherein the first container, the second container, and/or the third container comprises a fluid conduit, and the first connector, the second connector, and/or the third connector is arranged to be attached to the fluid conduit.

28. The system of claim 12, wherein the second container comprises a septum, and the first connector, the second connector, and/or the third connector is arranged to be attached to the septum.

29. The system of claim 12, wherein:
the first connector, and/or the second connector each comprise a first piece and a second piece, one end of the first piece being arranged to be attached to the first container, a second container, and/or the third container, and one end of the second piece being arranged to be attached to the second container.

30. The system of claim 12, wherein the first connector or the second connector comprise a first piece and a second piece, and wherein the first connector or the second connector further comprises an intermediate piece having a first end and a second end, which are arranged to be attached to a second end of the first piece and/or a second end of the second piece.

31. The system of claim 12, wherein the first connector or the second connector each comprise a first piece and a second piece, the first container and the second container comprises a fluid conduit, and the first piece is arranged to be attached to the fluid conduit, and the second container comprises a septum, and the second piece is arranged to be attached to the septum.

32. The system of claim 12, wherein the first connector or the second connector comprise a first piece and a second piece, wherein the first piece and the second piece form the first sterilizable space, a second sterilizable space, and/or a third sterilizable space.

33. The system of claim 12, wherein:
the first connector or the second connector comprise a first piece and a second piece,
the first piece and the second piece comprise: one or more valves, one or more seals, and one or more ports.

34. A method for non-parallel processing of multiple cell cultures, the method comprising:
(i) providing the system for non-parallel random access manufacturing of cells of any one of claims 1-33, wherein the system comprises multiple cell culture vessels, each of which comprises a cell culture; and
(ii) performing manufacturing operations on one or more of the cell cultures in the multiple cell culture vessels,
wherein, operates automated, manually, or a combination thereof.

35. The method of claim 34, wherein the manufacturing operations of (ii) comprise a connection interface for sterile connection and liquid transfer between one or more of the cell culture vessels and a bioprocess container.

36. The method of claim 35, wherein the sterile liquid transfer of the connection interface further comprises:
(a) placing a first tube and a second tube into a (coupling)mount, wherein the first tube is connected to a first container and the second tube is connected to a second container;
(b) welding(coupling) the first tube and the second tube to form a first sterile fluidical connection between the first container and the second container;
(c) transferring a liquid between the first container and the second container via the first sterile fluidical connection;
(d) sealing and cutting the first fluidical connection between the first container and the second container to disconnect the first sterile fluidical connection.

37. The method of claim 36, wherein the method further comprises:
(e) placing a third tube into the welding mount, wherein the third tube is connected to a third container;
(f) welding the first or second tube and the third tube to form a second sterile fluidical connection between the first container and the third container or between the second container and the third container;
(g) transferring a liquid between the first container and the third container or between the second container and the third container via the second sterile fluidical connection; and
(h) sealing the second fluidical connection between the connected first and third containers or between the connected second and third containers to disconnect the second sterile fluidical connection.

38. The method of claim 34, wherein welding the first tube to the second tube and/or the first or second tube to the third tube comprises welding a fresh portion of a tube of a pre-selected length in-between the first tube and the second tube, or in-between the first or second tube and the third tube, thereby connecting the first tube and second tube, or connecting the first or second tube to the third tube.

39. The method of claim 34, wherein step (ii) comprises performing the same manufacturing operation on multiple but not all cell cultures in the multiple cell culture vessels simultaneously or sequentially.

40. The method of claim 34, wherein step (ii) comprises performing different manufacturing operations on different cell cultures in the multiple cell culture vessels simultaneously or sequentially.
